(19) United States
(12) Plant Patent Application Publication
(10) Pub. No.: US 2002/0073467 P1
(43) Pub. Date: Jun. 13, 2002

(54) VARIETY OF GERANIUM PLANT NAMED 'FIWOCHER'

(76) Inventor: Andrea Michalik, Dresden (DE)

Correspondence Address: Russell D. Orkin, WEBB ZIESENHEIM LOGSDON ORKIN & HANSON, P.C., 700 Koppers Building, 436 Seventh Avenue, Pittsburgh, PA, 15219-1818, US

(21) Appl. No.: 09/733,021

(22) Filed: Dec. 8, 2000

(51) Int. Cl. A01H 5/00

(52) U.S. Cl. PLT/330

(57) ABSTRACT

A new and distinct zonal geranium plant with red flowers above medium green foliage. The plant flowers are suitable for planting in pots or as a bedding plant.

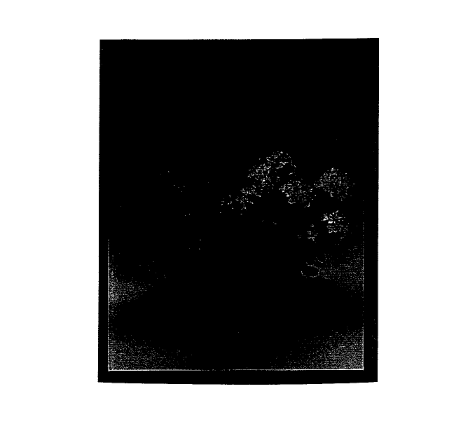

### BACKGROUND OF THE INVENTION

The present invention comprises a new and distinct cultivar of Pelargonium x hortorum known by the varietal name ‘Fiwocher’. The new variety was discovered in 1998 in a selective breeding program in Dresden, Germany. The new variety is the result of breeding of a group of about 60 stellar-type breeding varieties with red and white flowers. The new variety was first asexually reproduced by cuttings in Dresden, Germany in 1999. The new variety has been trial and field tested and has been found to retain its distinctive characteristics and remain true to type through successive propagations.

### DESCRIPTION OF THE DRAWING

The accompanying photographic drawing illustrates the new variety, with the color being as nearly true as is possible with color illustrations of this type.

### DESCRIPTION OF THE PLANT

The following detailed description sets forth the characteristics of the new cultivar. The data which defines these characteristics were collected by asexual reproductions carried out in Dresden, Germany. The color readings were taken outdoors in diffuse light. Color references are primarily to The R.H.S. Colour Chart of The Royal Horticultural Society of London.

Plant:

Classification.—Botanical: Pelargonium x hortorum. Commercial: Zonal geranium.

Form.—Erect.

Height from media surface to top of foliage.—50 cm.

Height from media surface to top of umbel.—65 cm.

Growth.—High degree of branching.

Strength.—Does not require artificial support.

Response time.—3.5 weeks.

Leaves.—Size: 6-10 cm. Shape: Stellar-type. Margin: Toothed. Texture: Smooth; some pubescence. Color: Green Group 139B with very weak zone. Ribs and veins: Color: Darker than leaf surface.

Petioles.—Color: Light green. Length: 3-5 cm.

Stem/branches.—Color: Medium green. Internode length: 3-5 cm.

The bud:

Shape when just showing color.—Overall: Semi-spherical. Individual bud: Narrow; elliptical.

Color.—Green Group 143B.

Size when just showing color.—Umbel: 8-10 cm across. Individual bud: 1-1.7 cm long; 0.5 cm wide.

Inflorescence:

Blooming habit.—Star-like; single.

Size of fully open bloom.—2.5-3.5 cm across.

Borne.—Flowers on umbel; umbel on pedicel; pedicel on peduncle.

Flowers.—Form: Zygomorph. Number of petals: 5. Size: Upper petals: 0.5-0.7 cm wide; 1.7 cm long. Lower petals: 0.7-1.0 cm wide; 1.7 cm long. Petals: Color: Upper surface: Red Group 52A. Lower surface: Red Group 52A. Eye: Present. Texture and appearance: Velvety.

Petaloids.—Absent.

Pedicel.—Length: 3-3.5 cm. Color: Medium green.

Peduncle.—Length: 15 cm. Color: Medium green.

Disease/pest resistance.—No unusual susceptibility to diseases or pests has been noted to date.

Lasting Quality.—Long-lasting; 3-4 weeks per umbel.

Reproductive organs:

Stamens.—Anthers: 3 mm long. Filaments: Length: 0.5 cm maximum. Color: White with greenish shine. Pollen: Dark yellow.

Pistils.—Number: Five. Length: 0.3-0.4 cm. Stigma: White in color. Style: 0.8 cm long.

Ovaries.—Color: Greenish. Length: 0.4 cm.

Fruit.—Present with 5 seeds; fruit production very weak.

### GENERAL CHARACTERISTICS

1. Stellar-shaped foliage.

2. Erect, compact growth.

3. Good branching.

4. Flowers single, star-like, two-colored.

5. Long-lasting with high resistance against rain. 6. Low care requirements.

I claim:

1. A new and distinct variety of geranium plant substantially as shown and described.
